(19) United States
(12) Patent Application Publication
(10) Pub. No.: US 2005/0064422 A1
(43) Pub. Date: Mar. 24, 2005

(54) Polypeptide quantitation

(76) Inventors: David R Barnidge, Rochester, MN (US); Arnold W Lindall, Stillwater, MN (US)

Correspondence Address: M Angela Parsons, Ph.D;Fsh & Richardson P.C.,P A., 60 South Sixth Street Suite 3300, Minneapolis, MN, 55402, US

(21) Appl. No.: 10/497,253

(22) PCT Filed: Nov. 27, 2002
(86) PCT No.: PCT/US02/38334

Related U.S. Application Data
(60) Provisional application No. 60/334,325, filed on Nov. 29, 2001.

(51) Int. Cl. C12Q 1/37

(52) U.S. Cl. 435/6; 435/23

(57) ABSTRACT

The invention relates to methods and materials useful for determining the actual amount of a selected polypeptide in a sample, by measuring the amount of a cleavage product released from the selected polypeptide and using an exogenous polypeptide corresponding to the cleavage product as a standard. These methods and materials can be used, for example, to quantify the actual amount of one or more selected polypeptides in complex samples.

### TECHNICAL FIELD

This invention relates to quantitative analysis of polypeptides. In particular, the invention pertains to methods and materials useful for determining the actual amount of a selected polypeptide in a sample. The methods involve measuring the amount of a specific cleavage product released from the selected polypeptide, with reference to an exogenous polypeptide that corresponds to the specific cleavage product.

### BACKGROUND

Polypeptides have important roles in biological systems. For example, polypeptides can function as enzymes that catalyze biological reactions, as transporters or carriers for a variety of molecules, as receptors for intercellular and intracellular signaling, as hormones, and as structural elements of cells, tissues and organs.

Determining the amount of a particular polypeptide is often important in research settings (e.g., in drug discovery and development) and in clinical settings (e.g., for medical diagnosis and for monitoring treatment efficacy). Particular polypeptides are commonly quantified by, for example, affinity methods, including immunoassays, mass spectrometry and high performance liquid chromatography. Radioisotopes, stable isotopes, fluorescence and chemiluminescence can be used in conjunction with these methods to quantify polypeptides. Enzymes have been quantified by biochemically assaying their catalytic activity.

Traditional methodologies can be limited in their ability to measure the actual, as opposed to relative, amount of a particular polypeptide in a sample. This shortcoming has made it difficult to evaluate changes in protein levels due, for example, to effects such as disease or therapeutic treatment. Quantitation of the actual amount of a particular polypeptide in a complex mixture or in a water insoluble environment (e.g., cell membrane) has proven to be particularly problematic. Therefore, the methods of the invention avoid many of the problems associated with insoluble proteins since a soluble proteolytic fragment can be chosen that quantitatively represents the intact protein.

### SUMMARY

The invention features methods and materials for determining the actual amount of a selected polypeptide in a sample. The methods involve measuring the amount of a specific cleavage product released from the selected polypeptide, with reference to an exogenous polypeptide that corresponds to the specific cleavage product. The disclosed methods and materials offer many advantages over traditional polypeptide quantitation methodologies. Actual, as opposed to relative, amounts of a selected polypeptide can be determined with reference to a readily made reference polypeptide. The reference polypeptide corresponds to the measured specific cleavage product, thereby eliminating errors related to differential behavior of the reference and the measured cleavage product. Measurement of membrane-associated proteins can be facilitated by releasing a specific cleavage product into solution (e.g., by targeting cleavage to solution-accessible sites in a selected polypeptide). The methods and materials of the invention can be used to quantitate the amount of one or more selected polypeptides, even in complex samples and in water insoluble environments.

The invention features methods for determining the actual amount of one or more selected polypeptides in a sample. The featured methods involve: 1) releasing at least one specific cleavage product from each selected polypeptide with at least one cleavage agent, and 2) determining the actual amount of each specific cleavage product by comparison to a defined amount of a corresponding exogenous polypeptide. The actual amount of each specific cleavage product is directly related to the actual amount of the selected polypeptide from which it was released.

In some embodiments, a sample contains one selected polypeptide. In other embodiments, a sample contains 2 to 5 selected polypeptides. In other embodiments, a sample contains 6 to 10 selected polypeptides.

In some embodiments, one specific cleavage product can be released from the selected polypeptide. In other embodiments, 2 to 5 specific cleavage products can be released from the selected polypeptide.

In some embodiments, there is a 1:1 direct relationship between the amount of the specific cleavage product and the selected polypeptide.

In some embodiments, a selected polypeptide is a membrane polypeptide. In some embodiments, a selected polypeptide is a neuroreceptor.

In some embodiments, the defined amount of the corresponding exogenous polypeptide is added to the sample prior to the releasing step.

In some embodiments, a cleavage agent is an enzyme (e.g., trypsin, endoproteinase Lys-C, endoproteinase Arg-C and endoproteinase Glu-C). In other embodiments, a cleavage agent is a chemical (e.g., cyanogen bromide).

In some embodiments, antibodies are used to measure the amount of a cleavage product. In other embodiments, tandem or higher order mass spectrometry is used to measure the amount of a cleavage product.

In some embodiments, the featured methods also involve: 1) adding a defined amount of a recovery polypeptide to a sample prior to releasing a cleavage product from a selected polypeptide, and 2) and measuring the actual amount of the recovery polypeptide after releasing the cleavage product from the selected polypeptide. In these embodiments, the actual amount of a specific cleavage product to which a recovery polypeptide corresponds is adjusted to reflect losses of the recovery polypeptide that occurred after the recovery polypeptide was added to the sample.

In some embodiments, the featured methods also involve: 1) adding a defined amount of a synthetic cleavable polypeptide to a sample prior to releasing a cleavage product from a selected polypeptide, 2) releasing at least two differentially labeled polypeptides from the synthetic cleavable polypeptide with the cleavage agent, and 3) measuring the actual amount of each differentially labeled cleavage product. In these embodiments, the actual amount of a specific cleavage product is adjusted to reflect incompleteness of cleavage. In some of these embodiments, one differentially labeled polypeptide corresponds to a specific cleavage product, and the actual amount of a specific cleavage product is adjusted to reflect losses of the corresponding differentially labeled polypeptide that occurred after the cleavable polypeptide was added to the sample.

Other features and advantages of the invention will be apparent from the following detailed description, and from the claims.

Unless otherwise defined, all technical and scientific terms used herein have the meaning commonly understood by one of ordinary skill in the art to which this invention belongs. All publications, patent applications, patents, and other references mentioned herein are incorporated by reference in their entirety. In case of conflict, the present specification, including definitions, will control. The disclosed materials, methods, and examples are illustrative only and not intended to be limiting. Skilled artisans will appreciate that methods and materials similar or equivalent to those described herein can be used to practice the invention.

### DESCRIPTION OF DRAWINGS

FIG. 1 shows an MS and MS/MS spectra of the synthetic polypeptide TETSQVAPA (SEQ ID NO:1).

FIG. 2 shows LC/MS/MS ion chromatograms for experimental and standard samples.

### DETAILED DESCRIPTION

The invention provides methods and materials for determining the actual amount of a selected polypeptide in a sample. The invention is based, at least in part, on the discovery that one can determine the actual amount of a selected polypeptide in a sample by releasing a specific cleavage product from a selected polypeptide, and then measuring the amount of the specific cleavage product with reference to an exogenous polypeptide that corresponds to the specific cleavage product. Without being bound by theory, this determination appears to be possible because there is a 1:1 molar relationship between the selected polypeptide and the released polypeptide that is measured. In addition, a specific cleavage product and a corresponding exogenous polypeptide behave the same way during measurement, thereby eliminating potential errors arising from differential behavior of the measured species and the reference species.

The provided methods and materials can be used to determine the actual amount of a single selected polypeptide in a sample, and can be used to determine the actual amount of multiple selected polypeptides in a sample. More than one specific cleavage product can be measured to increase sensitivity and/or check accuracy.

The methods disclosed herein are directly applicable to many current research needs in cell biology, protein chemistry, and clinical chemistry. The methods disclosed herein can provide absolute quantitation of a number of different polypeptides with greater sensitivity, dynamic range, precision, and speed than current methods offer. Because of the relatively short analytical cycle times required by the methods of the invention, the levels of a specific set of proteins can be measured kinetically at many time points after, for example, the onset of a stress or acute drug dosing to more clearly elucidate regulatory pathways. In addition, given that the present methods allow for a much shorter analytical cycle time than currently available methods, the methods of the invention are well suited for high-throughput experiments.

Selected Polypeptides and Samples

A selected polypeptide can be any polypeptide (i.e., 2 or more amino acids joined by a peptide bond), and a sample can be any polypeptide-containing sample. Suitable samples include cell samples, tissue samples, bodily fluids, and environmental samples. Samples can be derived from animals (e.g., humans) and can include animal cells, tissues or organs. Samples can be derived from plants and can include plant cells, tissues, or organs. Samples can also be derived from fungi, bacteria, and viruses. Samples can also be environmental (e.g. soil, water, and air samples). Polypeptides can be derived from animals, plants, fungi, bacteria, and viruses. Polypeptides can be membrane-associated (i.e., spanning a lipid bilayer or adsorbed to the surface of a lipid bilayer). Membrane-associated polypeptides can be associated with, for example, plasma membranes, cell walls, organelle membranes, and viral capsids. Polypeptides can be cytoplasmic or organeller. Polypeptides can be extracellular, being found interstitially or in bodily fluids (e.g., plasma, and spinal fluid). Polypeptides can be biological catalysts, transporters or carriers for a variety of molecules, receptors for intercellular and intracellular signaling, hormones, and structural elements of cells, tissues and organs. Some polypeptides are tumor markers.

Sample preparation is determined by the location and biophysical properties of the selected polypeptide and specific cleavage product to be measured. A sample can be enriched for the selected polypeptide before releasing specific cleavage products. Tissue or cell samples can be homogenized or left intact prior to treatment with a cleavage agent, depending on the cellular location of the selected polypeptide and the specific cleavage product to be measured. Membrane-associated polypeptides, such as receptors, are generally handled differently than cytoplasmic proteins. Cellular membranes can be isolated by, for example, centrifugation to enrich for membrane-associated polypeptides prior to treatment with a cleavage agent. Cytoplasm can be isolated during sample preparation to enrich for cytoplasmic proteins prior to treatment with a cleavage agent.

In some embodiments, samples are solubilized prior to treatment with a cleavage agent. Sample polypeptides can be solubilized in a variety of media, according to the nature of the sample. For example, a crude membrane preparation can be solubilized in a buffered detergent with 6 M urea, a reducing agent, and an alkylating agent. Samples can be defatted (e.g., in 95% alcohol and hexane or acetone) prior to treatment with a cleavage agent. Samples can be solubilized and defatted (e.g., in 95% alcohol and hexane or acetone) prior to treatment with a cleavage agent. In some instances, particularly where specific cleavage products are available in solution, samples can be digested without solubilizing or defatting. Specific cleavage products available in solution include, for example, cleavage products released from cytoplasmic, extracellular, interstitial, bodily fluid, and certain environmental polypeptides, as well as cleavage products released into solution from membrane-associated polypeptides.

Specific Cleavage Products and Cleavage Reactions

A specific cleavage product can be released from a selected polypeptide by treatment with one or more cleavage agents. This treatment can be accomplished via an in vitro or in situ cleavage reaction in which one or more cleavage agents are added to a polypeptide-containing sample. Cleavage agents cleave peptide bonds between particular amino acids in a polypeptide, and thereby release specific cleavage polypeptides. Cleavage agents can be used alone or in combination to release a specific cleavage product from a selected polypeptide. Some cleavage agents are enzymes, such as Endoproteinase Arg-C, Endoproteinase Glu-C, Endoproteinase Lys-C, and Trypsin. These particular endoproteinases are available from commercial vendors and have narrow specificity, making them ideal cleavage tools for use in protein quantitation. Other useful cleavage agents are chemicals, such as cyanogen bromide.

It is possible to predict the identity of the specific cleavage products that a cleavage agent will release from a selected polypeptide having a known amino acid sequence. Such a prediction is often referred to as a “virtual digest.” Readily available computer programs can facilitate preparation of a virtual digest of a selected polypeptide. A virtual trypsin digest of the rat purinergic receptor P2X3 (GenBank Accession No. CAA62594) is shown in Table 1. The end points of the specific cleavage products relative to amino acid positions in the native protein are indicated in the “from” and “to” columns.

TABLE 1

| | | | Amino Acid Sequences of Cleavage |
|---|---|---|---|
| Product | | | Products (letters represent the |
| # | From | To | single letter codes for amino acids) |
| 1 | 1 | 14 | MNCISDFFTYETTK (SEQ ID NO: 4) |
| 2 | 15 | 19 | SVVVK (SEQ ID NO: 5) |
| 3 | 20 | 28 | SWTIGIINR (SEQ ID NO: 6) |
| 4 | 29 | 47 | AVQLLIISYFVGWVFLHEK (SEQ ID NO: 7) |
| 5 | 48 | 52 | AYQVR (SEQ ID NO: 8) |
| 6 | 53 | 63 | DTAIESSVVTK (SEQ ID NO: 9) |
| 7 | 64 | 65 | VK |
| 8 | 66 | 69 | GFGR (SEQ ID NO: 10) |
| 9 | 70 | 73 | YANR (SEQ ID NO: 11) |
| 10 | 74 | 95 | VMDVSDYVTPPQGTSVFVIITK |
| | | | (SEQ ID NO: 12) |
| 11 | 96 | 113 | MIVTENQMQGFCPENEEK (SEQ ID NO: 13) |
| 12 | 114 | 115 | YR |
| 13 | 116 | 126 | CVSDSQCGPER (SEQ ID NO: 14) |
| 14 | 127 | 136 | FPGGGILTGR(SEQ ID NO: 15) |
| 15 | 137 | 145 | CVNYSSVLR (SEQ ID NO: 16) |
| 16 | 146 | 176 | TCEIQGWCPTEVDTVEMPIMMEAENFTIFIK |
| | | | (SEQ ID NO: 17) |
| 17 | 177 | 180 | NSIR (SEQ ID NO: 18) |
| 18 | 181 | 188 | FPLFNFEK (SEQ ID NO: 19) |
| 19 | 189 | 198 | GNLLPNLTDK (SEQ ID NO: 20) |
| 20 | 199 | 201 | DIK |
| 21 | 202 | 202 | R |
| 22 | 203 | 204 | CR |
| 23 | 205 | 209 | FHPEK (SEQ ID NO: 21) |
| 24 | 210 | 217 | APFCPILR (SEQ ID NO: 22) |
| 25 | 218 | 223 | VGDVVK (SEQ ID NO: 23) |
| 26 | 224 | 231 | FAGQDFAK (SEQ ID NO: 24) |
| 27 | 232 | 234 | LAR |
| 28 | 235 | 242 | TGGVLGIK (SEQ ID NO: 25) |
| 29 | 243 | 251 | IGWVCDLDK (SEQ ID NO: 26) |
| 30 | 252 | 259 | AWDQCIPK (SEQ ID NO: 27) |
| 31 | 260 | 264 | YSFTR (SEQ ID NO: 28) |
| 32 | 265 | 271 | LDGVSEK (SEQ ID NO: 29) |
| 33 | 272 | 281 | SSVSPGYNFR (SEQ ID NO: 30) |
| 34 | 282 | 284 | FAK |
| 35 | 285 | 287 | YYK |
| 36 | 288 | 295 | MENGSEYR (SEQ ID NO: 31) |
| 37 | 296 | 299 | TLLK (SEQ ID NO: 32) |
| 38 | 300 | 304 | AFGIR (SEQ ID NO: 33) |
| 39 | 305 | 315 | FDVLVYGNAGK (SEQ ID NO: 34) |
| 40 | 316 | 348 | FNIIPTIISSVAAFTSVGVGTVLCDIILLNFLK |
| | | | (SEQ ID NO: 35) |
| 41 | 349 | 354 | GADHYK (SEQ ID NO: 36) |
| 42 | 355 | 356 | AR |
| 43 | 357 | 357 | K |
| 44 | 358 | 367 | FEEVTETTLK (SEQ ID NO: 37) |
| 45 | 368 | 385 | GTASTNPVFASDQATVEK (SEQ ID NO: 38) |
| 46 | 386 | 397 | QSTDSGAYSIGH (SEQ ID NO: 39) |

Typically, specific cleavage products between 5 and 100 (e.g., 5-10, 10-20, 20-40, 60-80, and 80-100) amino acids are selected for measurement.

Typically, specific cleavage products that are likely to be released and accessible in solution are selected for measurement. Accessibility for cleavage and measurement can be evaluated, for example, on the basis of the known or predicted tertiary structure of the selected polypeptide. In addition, specific cleavage products having a relatively hydrophilic amino acid sequence are particularly suitable for measurement. The hydrophobicity/hydrophilicity of a specific cleavage product can be estimated using computer software, or manually on the basis of well known amino acid hydrophobicity indices.

Typically, specific cleavage products having low potential for post-translational modification are selected for measurement. Amino acid sequence determinants for post-translational modification are well known (See e.g., Han and Martinage, 1992, Int J Biochem., 24:19-28), and specific cleavage products lacking such sequence determinants are readily identified by manual inspection.

The conditions of a cleavage reaction are dependent on the cleavage agent used. Sample polypeptides can be diluted in a buffer containing any molecules that the cleavage agent requires for releasing specific cleavage products (e.g., ATP, or Mg²⁺). Treatment with a proteolytic enzyme typically is accomplished at an elevated temperature (e.g., 37° C.) for several hours or more.

Specific cleavage products can be obtained from a cleavage reaction by, for example, gel filtration, reverse phase chromatography (e.g., high performance liquid chromatography and fast performance liquid chromatography), solid phase extraction, ion exchange chromatography, affinity chromatography, and immunoaffinity separation, and by various combinations of these techniques. Antibodies useful for immunoaffinity separation can be made using exogenous peptides that correspond to a specific cleavage product.

Polypeptide Measurement and Quantitation

The actual amount of a specific cleavage product can be measured by any means known in the art. In some embodiments, the amount of a specific cleavage product is measured using mass spectrometry (e.g., tandem mass spectrometry or higher order mass spectrometry (e.g., MS^{N})). In other embodiments, the amount of a specific cleavage product is measured by an affinity assay such as an immunoassay (e.g., ELISA, or RIA). Immunoassays can be competitive or can be non-competitive. For measurement using RIA, exogenous polypeptides typically are used as tracers and typically are labeled with radioactive isotopes such as ³H, ¹⁴C, or ¹²⁵I. In other embodiments, the amount of a specific cleavage product is measured by high performance liquid chromatography. In some embodiments, measuring the actual amount of a specific cleavage product involves detectably labeling a cleavage product (e.g., by attachment of fluorescent, chemiluminescent, or radioactive molecules). For example, specific cleavage products can be labeled with stable isotopes such as ²H, N, ¹³C, or ¹⁸O for measurement using mass spectrometry.

The actual amount of a specific cleavage product is determined with reference to a corresponding exogenous polypeptide. Defined amounts of a corresponding exogenous polypeptide are measured and a standard curve relating the signal obtained to polypeptide quantity is created. Experimental samples are measured by the same means and a standard curve is used to translate the measured signal to actual polypeptide quantity. The actual amount of a selected polypeptide can be measured, in part, because a corresponding exogenous polypeptide behaves the same way as a specific cleavage product during measurement, thereby eliminating potential errors related to differential behavior of the specific cleavage product and the exogenous polypeptide. As used herein, the “actual” amount of a compound (e.g., a specific cleavage product, or a selected polypeptide) refers to the absolute amount of the compound in a sample. The “actual” amount of a compound can be obtained by direct measurements using, for example, mass spectrometry, or can be obtained by comparison to a standard curve produced using defined amounts of a corresponding compound. Such a corresponding compound is generally exogenous to the sample (i.e., originating or produced outside the cell, tissue, or organ). The “actual” or “absolute” amount of a compound can be contrasted with the “relative” amount of a compound, wherein the amount of the compound to be measured is based on or dependent upon (i.e., relative to) the amount of a compound that does not correspond (a “non-corresponding” compound) to the compound being measured. It would be apparent to those of ordinary skill in the art that a suitable non-corresponding compound should be the same type of compound as the compound being measured (e.g., if a polypeptide is being measured, the non-corresponding compound also should be a polypeptide).

The methods of the invention allow for determining the actual amount of a selected polypeptide because there is a 1:1 molar ratio between a polypeptide and a unique cleavage product generated therefrom. For the 1:1 ratio to hold true, complete cleavage by the cleavage agent is required. The invention further provides for methods to determine the actual amount of a selected polypeptide in cases where cleavage is incomplete (discussed below). The methods of the invention are particularly useful for obtaining actual amounts of polypeptides that, for example, are not readily or efficiently purified. Such polypeptides include, but are not limited to, membrane polypeptides (e.g., receptors such as G-protein coupled receptors and neuroreceptors). It can be appreciated that the methods of the invention can be used to determine the actual amount of the specific cleavage product(s) and, subsequently, the actual amount of the selected polypeptide(s), within a range of normal experimental error.

In applications that use mass spectrometry to quantitate a selected polypeptide, a corresponding exogenous polypeptide typically is compositionally identical to a specific cleavage product. As used herein, compositionally identical polypeptides contain the same amino acids, but may have a different primary sequence. In applications that use antibodies to quantitate a selected polypeptide, a corresponding exogenous polypeptide is specifically immunoreactive to an antibody that binds a specific cleavage product resulting from cleavage of the selected polypeptide. A specifically immunoreactive polypeptide is a polypeptide to which an antibody preparation binds and displays dilutional linearity (i.e., proportional reactivity over a series of antigen dilutions). An antibody preparation should not exhibit cross-reactivity with a polypeptide other than the selected polypeptide or a fragment therefrom. Specific immunoreactivity of an antibody preparation can be directed to any group of amino acids (e.g., an epitope) within a polypeptide. An antibody preparation specifically reactive to a polypeptide of one organism can be specifically immunoreactive to a structurally similar polypeptide of another organism. For example, an antibody preparation specifically reactive to a rat polypeptide can be specifically immunoreactive to a human polypeptide. Immunoreactive corresponding exogenous polypeptides should be identical to a specific cleavage product. It would be apparent to those of skill in the art that an antibody preparation used in an immunoassay to quantitate a selected polypeptide needs to be in excess in order that 100% of the specific cleavage product be detected.

Recovery Polypeptides and Cleavage Controls

A recovery polypeptide can be used to correct for any losses of a specific cleavage product that may occur during sample preparation, cleavage, and/or quantitative analysis. A recovery polypeptide is a labeled exogenous polypeptide that corresponds to a specific cleavage product. When used, a recovery polypeptide can be added in a defined amount directly to a sample as an internal control. Addition of a recovery polypeptide allows for correction for losses that may occur after the time at which it is added to a sample, and up to the time at which the recovery polypeptide is measured. Thus, to correct for all losses that occur during sample preparation, cleavage, and quantitative analysis, a defined amount of a recovery polypeptide can be added to a sample before beginning sample preparation, and the amount of the recovery polypeptide is then measured when the amount of the corresponding specific cleavage product is measured. Losses of the recovery polypeptide (and hence the corresponding specific cleavage product) can be determined by comparing the amount of the recovery polypeptide present after sample preparation to the defined amount added to the sample. The measured amount of the specific cleavage product can then be adjusted to reflect losses of the recovery polypeptide.

A recovery polypeptide can be identical to a measured specific cleavage product. In applications that use mass spectrometry to quantitate a selected polypeptide, a recovery polypeptide typically is identical to a measured specific cleavage product. In applications that use antibodies to quantitate a selected polypeptide, a recovery polypeptide typically is specifically immunoreactive to an antibody that binds a measured specific cleavage product.

Recovery polypeptides can be labeled using any means known in the art. For example, recovery polypeptides can be labeled with a stable isotope such as ²H, ¹⁵N, ¹³C and ¹⁸O for measurement using mass spectrometry. Recovery polypeptides can be labeled with ³H, ¹⁴C or ¹²⁵I for measurement using an immunoassay. Recovery polypeptides also can be labeled with fluorescent or chemiluminescent molecules. When a recovery polypeptide is added to a sample containing a labeled specific cleavage product, the labeled specific cleavage product and corresponding recovery polypeptide typically are differentially labeled. When a radioimmunoassay to measure a specific cleavage product is used, the amount of radioactivity in the recovery polypeptide typically is sufficiently low so as to not interfere with measurement of the specific cleavage product.

To verify a 1:1 molar relationship of specific cleavage product to the starting selected polypeptide, the completeness of a cleavage reaction should be verified. A variety of approaches can be undertaken to verify completion of a cleavage reaction. For example, a kinetic experiment that monitors the conversion of a known polypeptide (e.g., the selected polypeptide) to cleavage products can be used to estimate the time required for a particular cleavage agent to completely convert a selected polypeptide to cleavage products.

Another way to verify complete cleavage of a selected polypeptide involves the design and preparation of a differentially labeled synthetic cleavable peptide having a cleavable site (e.g., lysine, if the cleavage agent is trypsin). One or more amino acids on the N-terminal side of the cleavable site and one or more amino acids on the C-terminal side of the cleavable site are labeled with different isotopes. For example, amino acid(s) on the N-terminal side can be labeled with ¹⁴C and amino acid(s) on the C-terminal side of the cleavable site can be labeled with ³H. A differentially labeled cleavable polypeptide is added to a sample prior to the cleavage reaction, either directly to the sample containing a selected polypeptide, or to a parallel sample. The amount of isotopes can be quantitated using a two channel liquid scintillation counter, and the ratio of one isotope to the other is a measure of completeness of the cleavage reaction.

If a differentially labeled synthetic cleavable polypeptide is directly added to the sample containing a selected polypeptide, it is added in an amount that does not interfere with measurement of a specific cleavage product. If a cleavable peptide is directly added to sample containing a selected polypeptide, it is labeled differently than any labeled specific cleavage product (e.g., a cleavage product to be measured by MS/MS).

The amino acids on one or both sides of a cleavable site in a synthetic cleavable polypeptide can correspond to a specific cleavage product. If the amino acids on either side of the cleavable site correspond to a specific cleavage product to be measured, these amino acids can serve as a recovery polypeptide to account for any losses of the specific cleavage product.

Measurement of Multiple Cleavage Products

For any particular sample, a variety of different specific cleavage products can be measured. By measuring multiple specific cleavage products released from a particular selected polypeptide, one can increase the sensitivity and /or verify the accuracy of a quantitative analysis of that particular selected polypeptide. By measuring specific cleavage products released from different selected polypeptides, one can quantitate multiple selected polypeptides for a particular sample. Each specific cleavage product can be measured with reference to a corresponding exogenous polypeptide, and recovery polypeptides can be used to account for losses of any or all of the measured specific cleavage products.

The invention will be further described in the following examples, which do not limit the scope of the invention described in the claims.

### EXAMPLES

### example 1

### ELISA Quantitation of Human P2X₃

This example demonstrates the quantitation of human P2X₃, a membrane-associated purinoreceptor. A cleavage product released from the human P2X₃ carboxy terminus was measured with reference to a corresponding synthetic polypeptide. The cleavage product had the amino acid sequence: QSTDSGAFSIGH (SEQ ID NO:2). The corresponding synthetic polypeptide had the rat P2X₃ amino acid sequence: VEKQSTDSGAYSIGH (SEQ ID NO:3). The synthetic polypeptide was used to raise antibodies specifically immunoreactive to the synthetic polypeptide and to the cleavage product. The antibodies were also verified to demonstrate specific reactivity to the selected polypeptide by immunohistochemistry and Western blotting.

Samples and Cleavage Reactions: P2X₃-containing preparations were made from Hex cell transfectants expressing human P2X₃. Briefly, P2X₃-containing Hex cells were suspended in phosphate buffered saline (PBS), sonicated, and frozen at −70° C. Frozen cell suspensions were thawed in an ice bath, sonicated, and centrifuged at 100,000×g for 1 hour at 4° C. Pellets were resuspended in PBS. Cell suspensions were sonicated and centrifuged at 100,000×g for one hour at 4° C. Pellets were resuspended in membrane solubilization buffer (6 M urea, 2 mM dithiotreotol (DTT), 1% Chaps, 0.05 M Tris (pH 8.0)) so that the protein concentration in a membrane preparation was between about 1.0 to 1.5 mg per 100 μl. Protein determinations by the Pierce BCA method indicated that about 8 mg of total membrane protein were present in the samples. Western blotting confirmed the presence of P2X₃ in the membrane preparations. Membrane preparations were frozen at −70° C.

Before treatment with the cleavage reagent trypsin, cell suspensions were thawed, sonicated, incubated for 15 minutes at room temperature, and diluted seven-fold in 50 mM Tris (pH 7.6) and 1 mM MgCl₂ in 5 ml Wheaton vials. Cleavage reactions were initiated by the addition of 10 μg of trypsin (sequencing grade; Promega, Madison, Wis.) per 1 mg protein. Cleavage reactions were incubated with shaking at 37° C. for 24 hours. To confirm complete digestion, cytochrome C was digested with trypsin in parallel and the progress of digestion was monitored by HPLC. Cleavage reactions were acidified to about pH 1-2 with 100% TFA, and the resulting precipitate was removed by centrifugation at 10,000 rpm in a high-speed centrifuge.

Cleavage reactions were applied two times to a C18 Sep-Pak® (Waters Corp.) that had been washed with 10 ml methanol and then with 10 ml 0.1% TFA. The Sep-Pak® was washed with 1 ml 0.1% TFA and 8% acetonitrile. Cleavage products were eluted into a pre-weighed 12×75 mm tube with 1 ml 0.1% TFA and 48% acetonitrile. Eluates were dried under nitrogen to about 200 μl. By weighing the tube with reference to it's dry weight, the sample volume was brought to 300 μl with water, and a 200 μl aliquot was diluted 5× with 1% BSA-PBS and adjusted to pH 7.2-7.4 for ELISA. HPLC analysis confirmed nearly complete recovery of polypeptide, including cleavage products.

ELISA Measurement and Quantitation: A reference curve was generated as follows. 50 μl samples containing between 0.078 and 2.5 μg/ml synthetic polypeptide in BSA-PBS were pipetted into duplicate wells of a blocked, washed and blotted 96-well Nunc Polysorb plate, previously coated with 0.25 μg synthetic polypeptide per well. 50 μl of antibody (1/20,000 dilution) supplemented with trypsin inhibitor (Boehringer Mannheim, Indianapolis, Ind.) was added to each well. The antibodies were obtained from rabbits injected with the synthetic rat P2X₃ polypeptide VEKQSTDSGAYSIGH (SEQ ID NO:3) coupled to bovine thyroglobulin, and were confirmed by immunohistochemistry and Western blotting to be specifically reactive to both the human P2X₃ cleavage product QSTDSGAFSIGH (SEQ ID NO:2) and to the synthetic polypeptide VEKQSTDSGAYSIGH (SEQ ID NO:3). After 24-48 hours incubation at 4° C., the plate was washed four times and inverted on blotting paper. 100 μl of a Donkey anti-rabbit antibody (1/50,000 dilution) conjugated to horseradish peroxidase was added to each well, and after a 45-minute incubation at room temperature, the plate was washed 4 times and inverted on blotting paper. 100 μl HRP-substrate color reagent (500 μl 0.48% TMB solution +10 ml 0.1 M citrate buffer containing 0.0024 M H₂O₂ (pH 4.25)) was added to each well, and incubated until blue color was well developed. 100 μl 2.0 N H₂S0₄ was added to each well and absorbance at 450 nm was measured. Reference curves plotting A₄₅₀ as a function of the amount of VEKQSTDSGAYSIGH (SEQ ID NO:3) indicated a minimal detectable dose of about 1 pmol. Analysis of experimental samples indicated linearity of the assay as applied to the QSTDSGAFSIGH (SEQ ID NO:2) cleavage product.

The ELISA assay was used to measure the amount of cleavage product present in experimental trypsin-digested samples. Experimental ELISA measurements were compared to the reference curve to determine the amount of QSTDSGAFSIGH (SEQ ID NO:2) present in trypsin-digested samples. See Table 2.

TABLE 2

| | | | |
|---|---|---|---|
| μl assayed | pmol/well | pmol × dilution | total pm |

P2X₃-containing cells

| | | | |
|---|---|---|---|
| 50 | 9.85 | 9.85 | 295.5 |
| 25 | 4.39 | 8.78 | 263.4 |
| 12.5 | 2.30 | 9.2 | 276 |
| 6.25 | 0.99 | 7.92 | 237.6 |
| mean | | | 268.13 |

Untransfected Hex cells

| | | | |
|---|---|---|---|
| 50 | 1.1 | 1.1 | 33 |

To arrive at a quantitative amount of P2X₃, the measured amount of the cleavage product was adjusted to correct for losses that occurred during sample preparation, digestion, and processing. To control for losses, the synthetic polypeptide VEKQSTDSGAYSIGH (SEQ ID NO:3) was carried through parallel sample preparation, digestion, and processing steps. Recovery of the synthetic polypeptide was determined to be 67%. Table 3 shows the amount of P2X₃ present in P2X₃-containing Hex cells as determined in the above-described experiment, and in a replicate experiment.

TABLE 3

| | Experiment | Replicate |
|---|---|---|
| pmol/mg membrane protein | 50.0 | 71.1 |
| μg receptor/mg membrane protein | 3.00 | 4.27 |
| P2X₃ as % membrane protein | 0.30 | 0.43 |

### Example 2

### LC/MS/MS Quantitation of Rhodopsin

This example demonstrates the quantitation of rhodopsin, a transmembrane G-protein coupled receptor. A cleavage product released from the rhodopsin carboxy terminus was measured with reference to an identical synthetic polypeptide. The cleavage product and corresponding exogenous polypeptide had the following amino acid sequence: TETSQVAPA (SEQ ID NO: 1).

Samples and Cleavage Reactions: Rhodopsin-containing preparations were made from bovine rod outer segments (ROS) by the method of Nemis and Dratz (1982, Methods Enzymol., 81:116-23, Packer, ed., Academic Press, New York, N.Y.). ROS preparations containing 13 μg/μl or 315 pmol/μl rhodopsin were diluted 13-fold and 20 μl samples were dispensed into microfuge tubes. Each sample to be quantitated contained 485 pmol of rhodopsin. Some samples were supplemented with synthetic polypeptide in an amount of 480 pmol (i.e., 12 μl of a 40 pmol/μl stock). Control samples contained buffer and 480 pmol synthetic polypeptide.

Before treatment with the cleavage agent trypsin, samples were brought to a volume of 200 μl with 50 mM Tris buffer (pH 8.0)+1 mM CaCl₂. Cleavage reactions containing 5 μl of a 1 μg/μl stock of trypsin to the samples were incubated overnight at 37° C. Cleavage reactions were acidified by adding neat TFA to a concentration of 1%, and were centrifuged at 20,000×g for 30 min to pellet remaining ROS. Cleavage reactions were concentrated by vacuum centrifugation using a SpeedVac to a volume of about 40 μl. 10 μl of sample was used for LC/MS/MS analysis.

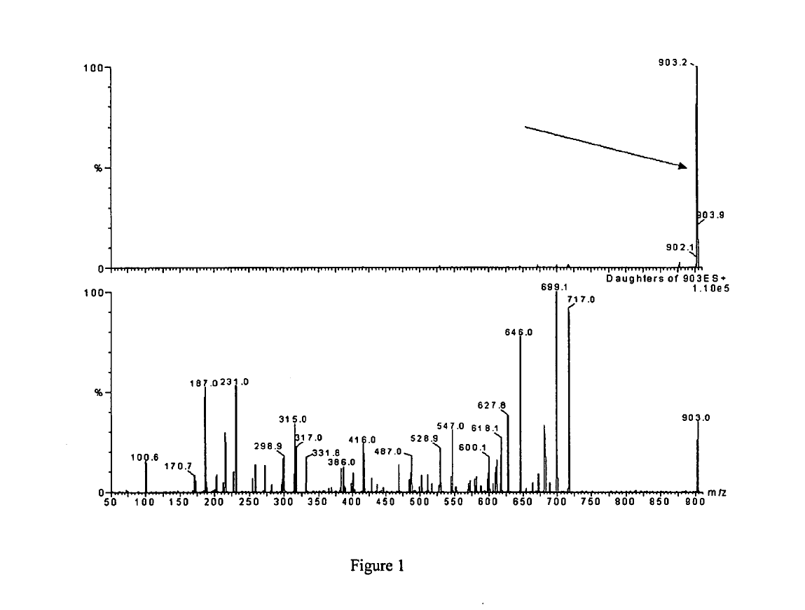

LC/MS/MS Measurement and Quantitation: A linear fit, 1× weighted reference curve was generated from LC/MS/MS measurements of the synthetic polypeptide TETSQVAPA (SEQ ID NO: 1) over a range of concentrations (i.e., 0.500 pmol/μl, 1 pmol/μl, and 40 pmol/μl). The reference curve was made using multiple reaction monitoring (i e., peak areas corresponding to multiple daughter ions derived from the singly- and doubly-charged synthetic polypeptide ion were determined). FIG. 1 shows MS and MS/MS spectra of the synthetic polypeptide TETSQVAPA (SEQ ID NO: 1). The top panel shows an MS spectrum, and the arrow indicates the peak representing the mass associated with the singly charged [M+H]⁺¹ ion of the polypeptide shown at m/z 903 Da. The bottom panel shows an MS/MS spectrum from the collisionally induced dissociation of the [M+H]⁺¹ ion of the polypeptide. Daughter ions used for LC/MS/MS quantitation were at m/z 717, 646, and 187. The reference curve indicated that the LC/MS/MS method had a detection limit of roughly 0.5 pmol, and a linear dynamic range of about 1 to 2000 pmol.

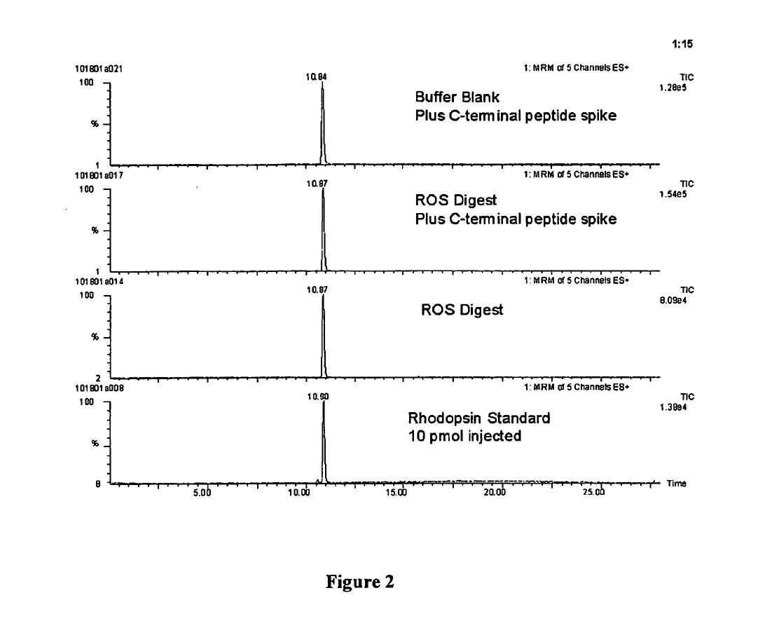

The LC/MS/MS method was used to measure the TETSQVAPA (SEQ ID NO: 1) polypeptide in experimental trypsin-digested samples containing ROS, ROS+synthetic polypeptide, and buffer+synthetic polypeptide. FIG. 2 shows LC/MS/MS ion chromatograms for experimental and standard samples. The areas of the peaks represent the number of polypeptide ions eluting from the HPLC column. Area counts from the peaks were used in the calculations for linear calibration curves and for determining the concentration of unknowns. Experimental LC/MS/MS measurements were compared to the reference curve to determine the amount of TETSQVAPA (SEQ ID NO: 1) present in trypsin-digested samples containing ROS, ROS+synthetic polypeptide, and buffer+synthetic polypeptide. See Table 4.

TABLE 4

| | Actual | Measured | Average | | |
|---|---|---|---|---|---|
| | Amount | Amount | Amount | CV | |
| | (pmol) | (pmol) | (pmol) | (%) | Recovery (%) |
| ROS | | | 161.7 ± 35.6 | 22.0 | 33.3 |
| Sample 1 | 485.0 | 145.3 | | | |
| Sample 2 | 485.0 | 137.4 | | | |
| Sample 3 | 485.0 | 202.6 | | | |
| ROS + | | | 421.6 ± 11.2 | 2.70 | 43.7 |
| Synthetic | | | | | |
| Polypeptide | | | | | |
| Sample 1 | 965 | 411.3 | | | |
| Sample 2 | 965 | 419.8 | | | |
| Sample 3 | 965 | 433.6 | | | |
| Buffer + | | | 234.9 ± 23.0 | 9.80 | 48.9 |
| Synthetic | | | | | |
| Polypeptide | | | | | |
| Sample 1 | 480 | 219.2 | | | |
| Sample 2 | 480 | 224.3 | | | |
| Sample 3 | 480 | 261.3 | | | |

The amount of the TETSQVAPA (SEQ ID NO:1) polypeptide measured in ROS samples is adjusted to account for the presence of supplemental synthetic TETSQVAPA (SEQ ID NO: 1) polypeptide, and is adjusted for recovery. The sample containing buffer and the synthetic TETSQVAPA (SEQ ID NO:1) polypeptide provides an indication of the amount of synthetic polypeptide present in the TETSQVAPA (SEQ ID NO: 1)-supplemented ROS samples after cleavage and measurement, and provides an indication of the recovery of the TETSQVAPA (SEQ ID NO: 1) polypeptide. It was assumed that the recovery of the synthetic polypeptide from buffer samples and ROS samples is similar.

For ROS samples supplemented with synthetic polypeptide: 421.6 pmol−234.9 pmol=186.7 pmol, and 186.7 pmol'(1/0.49)=381 pmol. The rhodopsin determination agrees well with the amount of rhodopsin known to be present in the sample (i.e., 381 pmol is 78.6% of 485 pmol). The cleavage product determination agrees well with the amount of the TETSQVAPA (SEQ ID NO: 1) cleavage products measured in unsupplemented ROS samples (i.e., 186.7 pmol versus 161.7 pmol). Thus, the assumption that the recovery of synthetic polypeptide from buffer samples and ROS samples is similar is most likely valid.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for determining the actual amount of one or more selected polypeptides in a sample, said method comprising:
releasing at least one specific cleavage product from each said one or more selected polypeptides with at least one cleavage agent; and
determining the actual amount of each said at least one specific cleavage product by comparison to a defined amount of a corresponding exogenous polypeptide,
wherein the actual amount of each said at least one specific cleavage product is directly related to the actual amount of the selected polypeptide from which it was released.

2. The method of claim 1, wherein said sample contains one selected polypeptide.

3. The method of claim 2, wherein one specific cleavage product is released from said selected polypeptide.

4. The method of claim 1, wherein said direct relationship between said specific cleavage product and said selected polypeptide is 1:1.

5. The method of claim 3, wherein said direct relationship between said specific cleavage product and said selected polypeptide is 1:1.

6. The method of claim 1, wherein said defined amount of said corresponding exogenous polypeptide is added to said sample prior to said releasing step.

7. The method of claim 2, wherein 2 to 5 specific cleavage products are released from said selected polypeptide.

8. The method of claim 1, wherein said sample contains 2 to 5 selected polypeptides.

9. The method of claim 8, wherein one specific cleavage product is released from each said selected polypeptide.

10. The method of claim 8, wherein 2 to 5 specific cleavage products are released from each said selected polypeptide.

11. The method of claim 1, wherein said sample contains 6 to 10 selected polypeptides.

12. The method of claim 11, wherein one specific cleavage product is released from each said selected polypeptide.

13. The method of claim 1 1, wherein 2 to 5 specific cleavage products are released from each said selected polypeptide.

14. The method of claim 1, wherein at least one of said cleavage agents is an enzyme.

15. The method of claim 14, wherein said enzyme is selected from the group consisting of: trypsin, endoproteinase Lys-C, endoproteinase Arg-C, and endoprot,einase Glu-C.

16. The method of claim 1, wherein at least one of said cleavage agents is a chemical.

17. The method of claim 16, wherein said chemical is cyanogen bromide.

18. The method of claim 1, wherein at least one of said selected polypeptides is a membrane polypeptide.

19. The method of claim 1, wherein at least one of said selected polypeptides is a neuroreceptor.

20. The method of claim 1, wherein said determining step comprises the use of antibodies to measure the actual amount of at least one of said cleavage products.

21. The method of claim 1, wherein said determining step comprises the use of tandem mass spectrometry or higher order mass spectrometry to measure the actual amount of at least one of said cleavage products.

22. The method of claim 1, further comprising:
adding a defined amount of a recovery polypeptide to said sample prior to said releasing step; and
measuring the actual amount of said recovery polypeptide after said releasing step,
wherein said determining step comprises adjusting the amount of one of said specific cleavage product(s) to which said recovery polypeptide corresponds to reflect losses of said recovery polypeptide that occurred after said adding step.

23. The method of claim 1, further comprising:
adding a defined amount of a synthetic cleavable polypeptide to said sample prior to said releasing step;
releasing at least two differentially labeled polypeptides from said synthetic cleavable polypeptide with said cleavage agent; and
measuring the actual amount of each said differentially labeled polypeptide,
wherein said determining step comprises adjusting the amount of each said specific cleavage product to reflect incompleteness of cleavage by said cleavage agent.

24. The method of claim 23, wherein one of said differentially labeled polypeptides corresponds to at least one said specific cleavage product(s), and wherein said determining step comprises adjusting the amount of said at least one specific cleavage product to reflect losses of said corresponding differentially labeled polypeptide that occurred after said adding step.
